(19) United States
(12) Patent Application Publication
(10) Pub. No.: US 2006/0177498 A1
(43) Pub. Date: Aug. 10, 2006

(54) Solid pharmaceutical composition comprising ramipril

(76) Inventors: Ramaswami Bharatrajan, Thane (West) (IN); Erich Zeisl, Wiesing (AU); Niklaus Kofler, Vomp (AU); Manisha Rajesh Patil, Thane (West) (IN); Parfulla S Sahasrabudhe, Mumbai (IN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/542,675

(22) PCT Filed: Jan. 21, 2004
(86) PCT No.: PCT/EP04/00456

§ 371 Date: Aug. 11, 2005

(30) Foreign Application Priority Data
Jan. 22, 2003 (GB) 0301471.9
Nov. 24, 2003 (DE) 103 54 862.9
Nov. 27, 2003 (NL) 1024899

(51) Int. Cl. A61K 9/20

(52) U.S. Cl. 424/464

(57) ABSTRACT

The present invention relates to solid pharmaceutical compositions comprising ramipril with a suitably low water content, and processes for preparing said compositions.

The present invention relates to solid pharmaceutical compositions comprising ramipril with a suitably low water content, and processes for preparing said compositions.

### DESCRIPTION OF THE INVENTION

The present invention relates to the discovery of stable pharmaceutical compositions containing ramipril and to methods for making such compositions.

Ramipril (1) corresponds to (2S,3aS,6aS)-1{-(S)-N-([(S)-1-carboxy-3-phenylpropyl]alanyl}octahydro-cyclopenta[b]pyrrole-2-carboxylic acid 1-ethyl ester and is used for the treatment of i.a. hypertension, heart failure, and nephropathia.

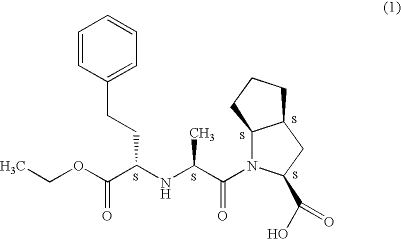

The preparation of ramipril has been described in EP 0 079 022 A2.

Stability is an important aspect of a pharmaceutical composition. The degradation of ramipril occurs mainly via two pathways: the hydrolysis to ramipril diacid [(2); Impurity E described in the European Pharmacopoeia] and the cyclization to ramipril diketopiperazide [(3): Impurity D described in European Pharmacopoeia].

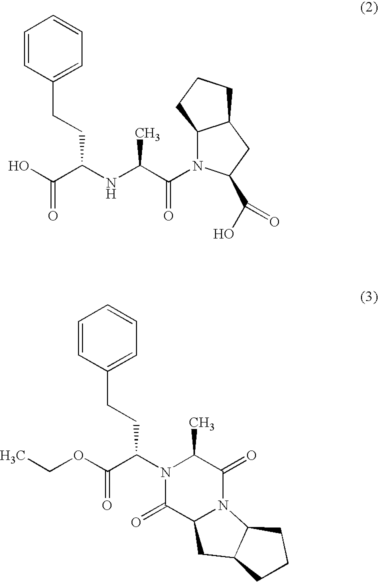

Information published in EP 0 317 878 A1, data generated on stress stability testing of commercial ramipril formulations (e.g. Delix®) and data generated internally during development of own formulations reveal that the major instability arises from the formation of the diketopiperazide. Table 1 shows the level of ramipril diketopiperazide and ramipril diacid after storage of Delix® 1.25 mg (batch number C423; originating from the German market) for 8 weeks at 40° C./75% relative humidity (RH).

TABLE 1

| ramipril diketopiperazide [%] | ramipril diacid [%] |
|---|---|
| 2.16 | 0.16 |

If not specified otherwise, assay values of ramipril (1), ramipril diacid (2) and ramipril diketopiperazide (3) in % are generated with suitable HPLC methods, e.g. as described in the European Pharmacopoeia 2001, monograph ‘Ramipril’.

The European Pharmacopoeia states and encourages a limit of 0.5% for diketopiperazide. Preferably the stability of a commercial composition is such that, after 3 months, preferably 6 months storage in a controlled environment of 40° C./75% RH, the loss of the active principle is less than 5% and the increase of impurities is preferably less than double the amount stated in the relevant Pharmacopoeia, in the case of ramipril the European Pharmacopoeia for the relevant impurity in the active principle. In the particular case of ramipril the level of ramipril diketopiperazide should preferably not exceed 1.0% after storage at 40° C./75% RH for 3 months, preferably 6 months.

According to EP 0 317 878 A1 it is well documented that ramipril formulations manufactured by standard technologies show a considerable degree of instability. Hoechst did manage to overcome the stability problem by applying a commercially expensive and technically complicated technology (coating of ramipril with a polymer prior to compression). It was surprisingly found that these prior art stability problems can be overcome applying pharmaceutical standard technologies when properly controlling/limiting water content in the final formulation. It was found that stability with the proposed formulations and processes is even improved over the currently marketed commercial formulations of ramipril. It was surprisingly found that other prior art approaches for stabilisation of ACE inhibitors (formulations with acid-donors, formulations with sodium bicarbonate) did not reveal a sufficiently stable formulation except when water content was properly controlled concurrently. In addition it was surprisingly found that testing formulations with controlled water content not applying prior art approaches prove sufficiently stable as well. Whereas the focus of the trials was put on tablet formulations, the principle could be demonstrated to be as well suitable for capsules and is considered to be suitable for sachets as well.

The cyclization of ramipril to the ramipril diketopiperazide seems to be directly linked to the presence of moisture in the formulation.

Therefore the invention covers a solid pharmaceutical composition containing
(a) ramipril and/or a pharmaceutical acceptable salt thereof and
(b) one or more pharmaceutical excipients,
   wherein the composition has a suitably low water content.

Solid pharmaceutical compositions according to the invention include tablets, capsules, capsulets and sachets. Tablets may be suitably coated (film coated tablets, pills). Capsule formulations may cover both soft and hard capsules.

The form of the ramipril and/or a pharmaceutical acceptable salt thereof is not particularly limited and includes all pharmaceutically acceptable anhydrates, solvates, hydrates, crystalline and amorphous forms. The amount of ramipril in the solid pharmaceutical composition is not particularly limited and comprises any amount that is pharmaceutically effective.

Low water content can be achieved by a combination of suitable excipients showing low water content, process parameters that prohibit uptake of moisture during manufacture and proper packaging material that prohibits uptake of moisture during storage of the finished dosage form over shelf life. Suitable excipients with low water content are most preferably special grades of microcrystalline cellulose (e.g. Avicel PH 112), starch (e.g. Starch 1500 LM), silicon dioxide (e.g. Syloid AL-1 FP), calcium hydrogen phosphate (e.g. Dicafos A) but should not be limited to the excipients mentioned herein but extended to all declared low water content excipients including diluents, binders lubricants, disintegrants colorants, etc. In another embodiment of the invention, one or more of the excipients can be dried prior to use or throughout the manufacturing process to achieve the required level of water content. Even when applying excipients with low levels of water the blend and final formulation is susceptible to take up moisture during manufacture and during storage. Accumulation of humidity during processing can be properly limited by performing the manufacture under controlled environmental conditions. Preferred is the manufacture in an environment of equal or less than 35% RH at ambient temperature, preferably in an environment of equal or less than 35% RH at equal or less than 30° C.

Accumulation of moisture during storage can be properly avoided by using packaging materials known to be suitably tight against penetration of humidity. Preferred packaging materials are containers including lid composed of polyethylene and/or polypropylene and/or glass, and blisters or strips composed of aluminium or high density polyethylene.

Therefore, other embodiments of the inventions are packages comprising compositions of a suitably low water content packaged with packaging materials which are suitably tight against penetration of humidity, preferably packaging materials as mentioned above.

The water content in the composition can, for example, be determined by loss-on-drying (LOD) and/or Karl-Fischer (KF)-analysis as it is understood by workers skilled in the art. For the determination of all the data cited the below mentioned methods were used. Out of these two methods, KF is known to be more reproducible and specific. Thus KF is the preferred method to assess water content in pharmaceutical formulations.

LOD: For tablet formulations, tablets are crushed to powder in a mortar with a pestle. For capsule or sachet formulations, the content of the capsule or sachet is emptied. The loss on drying is determined on a moisture balance e.g. Mettler LP 16 using approximately 1.0 g of the sample. The mixture is evenly spread on the weighing plate of the moisture balance. The weighing plate is preheated to 80° C. and the mixture is then dried for 15 minutes at 80° C.

KF: For tablet formulations tablets are crushed to powder in a mortar with a pestle. For capsule or sachet formulations the content of the capsule or sachet is emptied out. The water content is determined with an automated KF apparatus e.g. Metrohm 784 KFP Titrino using conventional Karl Fischer reagent using 0.1 g of the sample.

Manufacturing the product with conventional excipients results in a considerably high decrease of ramipril and increase in ramipril diketopiperazide on storage. In EP 0 317 878 A1. the increase of ramipril diketopiperazide up to 22.8% after 6 months at 40° C./170% RH and the decrease of ramipril down to 20% after 6 months at 40° C. was attributed to mechanical stress, and therefore the active principle was coated in order to protect it from mechanical stress.

Commercial formulations of Ramipril can be taken as reference for formulations manufactured with conventional excipients and showing normal levels of water content. Results on commercial formulations of Ramipril achieved with LOD and KF are presented in Tables 2 and 3.

TABLE 2
Water content of originator formulation as determined by KF

| | | | | | | Water |
|---|---|---|---|---|---|---|
| Product | Batch | | | | | content |
| Name | No. | Strength | | Formulation | Country | [weight-%] |
| Delix ® | 40A428 | 2.5 | mg | Tablets | Germany | 8.00 |
| Delix ® | 40A475 | 5 | mg | Tablets | Germany | 7.34 |
| Delix ® | 1W425 | 10 | mg | Tablets | Germany | 7.24 |
| Protect | | | | | | |
| Tritace ® | W245 | 1.25 | mg | Capsule | Austria | 11.07 |
| Tritace ® | A232 | 2.5 | mg | Capsule | Austria | 10.03 |
| Tritace ® | A228 | 5 | mg | Capsule | Austria | 9.95 |
| Tritace ® | W429 | 10 | mg | Tablets | Austria | 7.78 |

TABLE 3
Water content of originator formulation as determined by LOD

| | | | | | | Water |
|---|---|---|---|---|---|---|
| Product | Batch | | | | | content |
| Name | No. | Strength | | Formulation | Country | [weight-%] |
| Delix ® | 40A428 | 2.5 | mg | Tablets | Germany | 6.37% |
| Delix ® | 40A475 | 5 | mg | Tablets | Germany | 6.11% |
| Delix ® | 1W425 | 10 | mg | Tablets | Germany | 5.97% |
| Protect | | | | | | |
| Tritace ® | W245 | 1.25 | mg | Capsule | Austria | 9.52% |
| Tritace ® | A232 | 2.5 | mg | Capsule | Austria | 8.68% |
| Tritace ® | A228 | 5 | mg | Capsule | Austria | 8.40% |
| Tritace ® | W429 | 10 | mg | Tablets | Austria | 6.70% |

This type of formulation is only stable when ramipril is separated by a polymeric barrier from the water-containing excipients. The effect of the barrier was attributed by EP 0 317 878 A1 to reduced mechanical stress during compression, but according to our surprising findings might as well be attributable to minimising the contact of ramipril with water on storage over the shelf life.

Stability results generated on Examples 3 and 4 demonstrate the superior stability of formulations with low water contents (Table 4).

TABLE 4
Stability of ramipril in tablets as a function of water content
Assay of ramipril [%]

| | Example 3 (water content by | Example 4 (water content by |
|---|---|---|
| | LOD: 2.71 weight-%) | LOD: 6.60 weight-%) |
| Initial | 101.11 | 97.85 |
| 1 week | 102.23 | 90.73 |
| 2 weeks | 99.90 | — |
| 4 weeks | 101.21 | — |

Besides choosing excipients with low water content, performing the manufacture in an environment of sufficiently low relative humidity is essential as demonstrated in enclosed example. A blend manufactured according to Example 1 was exposed to well defined environmental conditions of relative humidity at ambient temperature for up to 6 hours. Only when maintaining the relative humidity at approximately 30% the initial load with moisture could be maintained. At ambient humidity levels (50-60%) the blend has significantly taken up moisture already after 2 hours (Table 5). Considering that normal processing times for pharmaceutical products range from 8 hour up to one week control of this parameter becomes essential.

TABLE 5
Water uptake in tablets as a function of relative humidity during production

| | 30% RH | | 50-60% RH | | 70% RH | |
|---|---|---|---|---|---|---|
| | LOD | KF | LOD | KF | LOD | KF |
| Time [h] | [weight-%] | [weight-%] | [weight-%] | [weight-%] | [weight-%] | [weight-%] |
| 2 | 3.21 | 4.09 | 5.31 | 6.60 | 5.79 | 6.87 |
| 4 | 3.19 | 5.33 | 5.41 | 6.62 | 7.49 | 7.70 |
| 6 | 3.51 | 4.24 | 5.91 | 6.67 | 7.90 | 8.46 |

The third factor to control humidity in the final product is to prevent uptake of moisture during storage. It is well established that storing products in the containers including lid made of polypropylene and/or polyethylene and/or glass or in blisters and/or strips composed of aluminium or high density polyethylene prevents them from taking up moisture during storage over shelf life. Table 6 shows the uptake of moisture of tablets manufactured according to Example 1 and stored at 40° C./75% RH in various packaging materials. Whereas trilaminate (PVC/PE/PVDC 250μ/25μ/90 gsm and aluminium foil 20 μm) blister packs reach a level of saturation already after 1 month, polypropylene containers with polyethylene lid and Alu/Alu strips show no increase in water content over up to 6 months.

TABLE 6
Water content as a function of packaging material

| | LOD [weight-%] | | |
|---|---|---|---|
| | PVC/PE/PVDC | | Polypropylene |
| | 250 μ/25 μ/90 gsm and | | container |
| Time | aluminium foil 20 μm | Alu/Alu 40 | with poly- |
| [months] | blister pack | μm strip pack | ethylene lid |
| 0 | 3.29 | 3.29 | 3.29 |
| 1 | 5.70 | 3.51 | — |
| 2 | 5.79 | 2.61 | — |
| 3 | — | 2.30 | 2.81 |
| 6 | — | 1.93 | 2.79 |

Application of the above mentioned principles reliably yields compositions with a suitably low water content, preferably less than 4.0 weight-%, most preferably less than 3.0 weight-% as determined by LOD, or less than 5.5 weight-%, most preferably less than 4.5 weight-% as determined by KF. By applying adequate packaging technologies the moisture content in the formulation can be maintained adequately low.

It was surprisingly observed that these pharmaceutical compositions only prove sufficiently stable under accelerated testing conditions when water content is low. Concurrently with increase of moisture, degradation to diketopiperazide occurs.

### WORKING EXAMPLES

### Example 1

During the manufacturing process environmental conditions of 30% RH/30° C. are kept. Milled glycine hydrochloride (0.300 kg) is dry-mixed with ramipril (0.125 kg), microcrystalline cellulose (Avicel PH112; 7.125 kg), precipitated silicon dioxide (Syloid AL-1-FP; 0.800 kg) and pregelatinised starch (Starch 1500 LM; 0.450 kg), and the resulting mixture is dry-mixed with glycerol dibehenate (Compritol ATO 888; 0.200 kg) and compressed to yield 100,000 tablets containing 1.25 mg Ramipril each.

For enclosed stability investigation the tablets are immediately packaged into PVC/PE/PVDC 250μ/25μ/90 gsm and aluminium foil 20 μm blister packs and Alu/Alu 40 μm strips. The samples are subjected to stability testing at 40° C./75% RH. The LOD of the tablets after manufacture is 3.19 weight-%.

TABLE 7
Stability and water content as a function of packaging material

| | | | | PVC/PE/PVDC 250μ/25μ/90 gsm and | | |
|---|---|---|---|---|---|---|
| Package | Alu/Alu 40 μm strip pack | | | aluminium foil 20 μm blister pack | | |
| Time | LOD | Ramipril | Diketopiperazide | LOD | Ramipril | Diketopiperazide |
| [months] | [weight-%] | [%] | [%] | [weight-%] | [%] | [%] |
| 0 | 3.19 | 98.68 | 0.12 | 3.19 | 98.68 | 0.12 |
| 2 | 2.91 | 97.87 | 0.12 | 5.81 | 65.63 | 9.68 |

Example 1 demonstrates that pharmaceutical compositions prepared with glycine hydrochloride prove sufficiently stable under accelerated testing conditions when water content is low but prove unstable when water content increases to normal levels of moisture within pharmaceutical formulations.

### Example 2

During the manufacturing process environmental conditions of 30% RH/30° C. are kept. Milled glycine hydrochloride (0.300 kg) is dry-mixed with Ramipril (0.500 kg), microcrystalline cellulose (Avicel PH112; 29.36 kg), precipitated silicon dioxide (Syloid AL-1 FP; 3.200 kg), pregelatinised starch (Starch 1500 LM; 1.800 kg), and Iron Oxide Red (0.040 kg) and the resulting mixture is dry-mixed with glycerol dibehenate (Compritol ATO 888; 0.800 kg) and compressed to yield 100,000 tablets containing 5 mg ramipril each, which are immediately packaged into PVC/PE/PVDC 250μ/25μ/90 gsm and aluminium foil 20 μm blister packs, Alu/Alu 40 μm strips and polypropylene container with polyethylene lid. The LOD of the tablets after manufacture is 3.19 weight-%.

TABLE 8
Stability of ramipril (1), generation of ramipril diketopiperazide
(3) and water content as a function of packaging material

| | PVC/PE/PVDC | | | | | | | | |
|---|---|---|---|---|---|---|---|---|---|
| | 250μ/25μ/90 gsm and | | | | | | | | |
| | aluminium foil 20 μm | | | | | | Polypropylene container | | |
| Package | blister pack | | | Alu/Alu 40 μm strip pack | | | with polyethylene lid | | |
| Time | LOD | (1) | (3) | LOD | (1) | (3) | LOD | (1) | (3) |
| [months] | [weight-%] | [%] | [%] | [weight-%] | [%] | [%] | [weight-%] | [%] | [%] |
| 0 | 2.41 | 99.78 | 0.14 | 2.41 | 99.78 | 0.14 | 2.41 | 99.78 | 0.14 |
| 1 | 3.97 | 99.56 | 0.09 | 1.90 | 100.88 | 0.12 | — | — | — |
| 2 | 4.71 | 92.54 | 3.89 | 2.11 | 101.61 | 0.18 | — | — | — |
| 3 | — | — | — | 1.50 | 99.34 | 0.16 | 2.19 | 99.43 | 0.15 |
| 6 | — | — | — | | | | | | |

Example 2 supports the findings of example 1. In particular example 2 demonstrates that low humidity compositions maintain levels of diketopiperazide far below the limit of 0.5% as stated in the European Pharmacopoeia and far below the results obtained for commercial ramipril formulation (Delix® 1.25 mg batch number C-423; originating from the German market; 2.16% diketopiperazide).

### Example 3

In analogy to example 1 aluminium strips containing tablets with the following composition are prepared: ramipril (1.25 mg), microcrystalline cellulose (Avicel PH112; 50.32 mg), precipitated silicon dioxide (Syloid AL-1-FP; 4.6 mg), lactose (Lactose DCL-21; 37 mg), glycerol dibehenate (Compritol ATO 888; 1.83 mg) at laboratory scale at ambient environmental conditions. The tablets are packaged into Alu/Alu 40 μm strips and put on stability at 40° C./75% RH. The LOD of the tablets after manufacture is 2.71 weight-%.

TABLE 9
Stability at low water content

| | ramipril [%] |
|---|---|
| Initial | 101.11 |
| 1 week | 102.23 |
| 2 weeks | 99.90 |
| 4 weeks | 101.21 |

Example 3 demonstrates that pharmaceutical compositions prepared with suitable excipients and not containing prior art stabilising agents do not show any significant degradation over 4 weeks when stored under accelerated testing conditions.

### Example 4

In analogy to example 1 aluminium strips containing tablets with the following composition are prepared: ramipril (1.25 mg), starch (Starch 1500; 20.32 mg), silicon dioxide (Aerosil 200; 1.00 mg), lactose (Lactose DCL-21; 78.00 mg), Ac-Di-Sol (4.00 mg) and Sterotex (1.80 mg) at laboratory scale at ambient environmental conditions. The tablets are packaged into Alu/Alu 40 μm strips and put on stability at 40° C./75% RH. The LOD of the tablets after manufacture is 6.60 weight-%.

TABLE 10
Stability at high water content

| | ramipril [%] |
|---|---|
| Initial | 97.85 |
| 1 week | 90.73 |
| 2 weeks | — |
| 4 weeks | — |

Example 4 demonstrates that pharmaceutical compositions prepared with conventional excipients and not containing prior art stabilising agents do not prove stable when stored under accelerated testing conditions. Already after one week of storage the content of ramipril has decreased by more than 5%.

### Example 5

At laboratory scale at ambient environmental conditions capsules containing ramipril (1.25 mg) and starch (Starch 1500; 138.75 mg) are prepared by dry-mixing of ramipril and Starch 1500 and filling the blend into conventional hard gelatine capsules. The capsules are packaged into Alu/Alu 40 μm strips and put on stability at 40° C./75% RH. The LOD of the capsules is 8.27 weight-%.

TABLE 11
Stability at high water content

| | Diketopiperazide [%] |
|---|---|
| Initial | 0.15 |
| 1 week | 0.81 |
| 2 weeks | — |
| 4 weeks | 2.14 |
| 6 weeks | 3.93 |

Example 5 demonstrates that capsule formulations showing a conventional level of water content do not prove stable when stored under accelerated testing conditions. After 6 weeks of storage at accelerated testing conditions the content of diketopiperazide has increased up to 4%.

### Example 6

In analogy to example 5 aluminium strips containing capsules with the following composition are prepared: ramipril (1.25 mg), starch (Starch 1500 LM; 37.00 mg) and perlitol (148.75 mg) are mixed and the blend is filled into conventional capsules at laboratory scale at ambient environmental conditions. The capsules are packaged into Alu/Alu 40 μm strips and put on stability at 40° C./75% RH. The LOD of the capsules 5.79 weight-%.

TABLE 12
Stability at high water content

| | Diketopiperazide [%] |
|---|---|
| Initial | 0.19 |
| 1 week | 0.81 |
| 2 weeks | — |
| 4 weeks | 1.23 |
| 6 weeks | 3.01 |

Example 6 support the findings of example 5. A LOD above 5 weight-% does not allow a sufficiently stable formulation. A significant trend toward stabilisation with decreased moisture load is obvious.

### Example 7

In analogy to example 5 aluminium strips containing capsules with the following composition are prepared: ramipril (1.25 mg), microcrystalline cellulose (Avicel PH 101; 71.48 mg), starch (Starch 1500; 20.47 mg), and arginine (1.80 mg) are mixed and the blend is filled into conventional capsules at laboratory scale at ambient environmental conditions. The capsules are packaged into Alu/Alu 40 μm strips and put on stability at 40° C./75% RH. The LOD of the capsules 3.24 weight-%.

TABLE 13
Stability at low water content

| | Diketopiperazide [%] |
|---|---|
| Initial | 0.71 |
| 1 week | 0.23 |
| 2 weeks | — |
| 4 weeks | 0.32 |

Example 7 demonstrates that capsule formulations showing a low level of water content prove considerably stable towards cyclization of the active principle to diketopiperazide when storage at accelerated testing conditions. Example 5, 6 and 7 demonstrate that the principle of stabilising ramipril formulations by excluding moisture in the formulation applies for capsule formulations as well. The assay of ramipril as well remains above 95%.

1. Solid pharmaceutical composition comprising
(a) an effective amount of ramipril and/or a pharmaceutical acceptable salt thereof and
(b) one or more pharmaceutically acceptable excipients,
characterized in that the composition is stabilized by having a suitably low water content of less than about 4.0 weight-% measured by loss-on-drying or of less than about 5.5 weight-% measured by Karl-Fischer-analysis.

2. Composition according to claim 1, wherein the water content is less than about 4.5 weight-% measured by Karl-Fischer-analysis.

3. Composition according to claim 1, wherein the water content is less than about 3.0 weight-% measured by loss-on-drying.

4. Composition according to claim 1, wherein ramipril and/or a pharmaceutical acceptable salt thereof is in form of pharmaceutically acceptable anhydrate, solvate and/or, hydrate and/or in crystalline and amorphous form.

5. Composition according claim 1, wherein the pharmaceutical composition is a tablet.

6. Composition according to claim 5, wherein the tablet is coated to generate a film coated tablet and/or a pill.

7. Composition according to claim 1, wherein the pharmaceutical composition is a capsule.

8. Composition according to claim 1, wherein the pharmaceutical composition is a sachet.

9. Composition according to claim 1, wherein the excipients have a suitably low water content.

10. Composition according to claim 9, wherein one of said excipients is microcrystalline cellulose.

11. Composition according to claim 1, wherein said excipients are selected from the group consisting of Avicel PH 112, starch, Starch 1500 LM, silicon dioxide, Syloid AL-1 FP, calcium hydrogen phosphate, Dicafos A or A Tab, anhydrous Emcompress, lactose, Pharmatose DCL 21, mannitol, Perlitol, calcium sulphate. Destab, Drierte, and mixtures thereof.

12-23. (canceled)

24. Composition according to claim 1 where one or more excipients are dried prior to use or throughout the manufacturing process to achieve the required level of water content.

25. Process for the preparation of a composition according to claim 1, wherein environmental conditions during manufacture are maintained at a relative humidity equal or less than 35% at ambient temperature.

26. Process for the preparation of a composition according to claim 1, wherein environmental conditions during manufacture are maintained at a relative humidity equal or less than 30° C.

27. Process according to claim 1, wherein the pharmaceutical composition is packaged into a packaging material suitably tight against penetration of humidity.

28. Process according to claim 27, wherein the packaging material is a container including lid composed of polyethylene and/or polypropylene and/or glass.

29. Process according to claim 27, wherein the packaging material is a strip or blister pack composed of aluminium which might be suitably coated or high density polyethylene.

30. Package comprising a composition according to claim 1 packaged with packaging material suitably tight against penetration of humidity.

31. Package according to claim 30, wherein the packaging material is a container including lid composed of polyethylene and/or polypropylene and/or glass.

32. Package according to claim 30, wherein the packaging material is a strip or blister pack composed of aluminium which might be suitably coated or high density polyethylene.
